(19) United States
(12) Patent Application Publication
(10) Pub. No.: US 2026/0275416 A1
(43) Pub. Date: Sep. 17, 2026

(54) CELL CONTAMINATION ASSAY

(71) Applicant: SISTEMIC SCOTLAND LTD, Glasgow (GB)

(72) Inventors: David Mallinson, Glasgow (GB); Elaine Gourlay, Glasgow (GB); James Reid, Glasgow (GB); Ditte Andersen, Glasgow (GB)

(73) Assignee: SISTEMIC SCOTLAND LTD, Glasgow (GB)

(21) Appl. No.: 19/675,717

(22) Filed: May 13, 2026

Related U.S. Application Data
(63) Continuation of application No. 16/963,325, filed on Jul. 20, 2020, now abandoned.

(51) Int. Cl. C12Q 1/6881; A61K 35/28; C12N 5/0775

(52) U.S. Cl. CPC C12Q 1/6881; A61K 35/28; C12N 5/0668; C12N 2506/45; C12Q 2600/158; C12Q 2600/178

(57) ABSTRACT

A method for determining the presence and level of PSC contaminants in a PSC-derived cell population for use in cell therapy by assaying a sample of the PSC-derived cell population against a panel of non-coding RNAs such as miRNA known to be differentially expressed in PSC contaminants, thereby detecting residual PSC cell contamination at a level of 10 and even 5 or fewer residual contaminating PSC cells in a background of one million cells, such that a PSC-derived cell population or sample may be identified as meeting safety requirements for use in cell therapy.

### RELATED APPLICATIONS

This application is a continuation of U.S. patent application Ser. No. 16/963,325, filed Jul. 20, 2020; that in turn is a 371 filing of PCT/EP2019/051530, filed Jan. 22, 2019; that in turn claims priority benefit of GB 1801035.5, filed 22 Jan. 2018 and GB1809337.7, filed Jun. 6, 2018; the contents of which are hereby incorporated by reference.

### FIELD OF THE INVENTION

This invention pertains generally to the cell contamination and cell therapies. More particularly, the invention relates to the determination of contamination in cell populations, assays for use in such determination, kits for use in such assays, cell populations and methods of treatment using such cell populations.

### BACKGROUND OF THE INVENTION

Stem cell-derived therapies have great promise in the effective treatment of many human diseases. They can be classified primarily into adult stem cells and cell therapy products derived from pluripotent stem cells (PSC). PSCs are stem cells that can differentiate into all of the cell types within the human body providing a renewable cell source as a basis of a derived cell therapies.

As the cell therapy sector develops, PSCs are becoming an increasingly popular way of producing derived cell therapy treatments and can be either embryonic stem cells (ESCs) or induced pluripotent stem cells (iPSCs).

The number of cell therapy products derived from PSCs in clinical trials have increased rapidly over recent years and this is anticipated to continue.

A common challenge with cell therapy products derived from PSCs is that residual undifferentiated PSCs in the derived cell product can give rise to teratomas or other neoplasms, which is a critical safety issue for such therapies.

As such, a key safety barrier to the application of these therapies is the regulatory requirement to be able to demonstrate and quantitatively assess the level of residual contaminating PSCs in the final clinical product. This poses a key challenge to the progression of PSC-derived cell therapies.

Currently there is no simple established way to assess the level of contamination by undifferentiated PSCs. The current options and approaches to address this issue are time consuming, lack the required sensitivity and are costly in vitro or in vivo assays, which are generally not suitable for routine quality control (QC) testing. In particular, there is currently no simple and rapid assay platform suitable for routine QC testing and lot release.

The availability of a simple, sensitive and quantitative assay suitable for routine QC testing and lot release, is crucial to the continued and successful clinical development of PSC-derived therapies.

MicroRNAs (miRNA) are single-stranded non-coding RNA molecules having a length of around 21 to 23 nucleotides. They regulate gene expression in cells by targeting specific ‘target’ gene products via hybridisation to mRNA transcripts, resulting in translational blockade or transcript degradation. Within a cell a single miRNA can regulate multiple genes (up to 1000s) and each gene can be regulated by multiple miR As. There are currently 2588 known human miRNAs which are estimated to form regulatory networks that regulate up to 60% of all human genes. These small regulators have important roles in multiple essential biological processes.

The present inventors have identified that microRNA (miRNA) expression or profile data can be used to determine levels of PSC contamination in PSC-derived cells in a simple, sensitive and quantitative assay.

### Problem to be Solved by the Invention

There is a need for improvements in the detection or determination of contamination by PSCs in PSC-derived cell populations for use in cell therapy.

It is an object of this invention to provide a method for detecting or determining contamination by PSCs in PSC-derived cell populations for use in cell therapy.

It is a further object of this invention to provide a method for quality control or lot release in PSC-derived cell populations for use in cell therapy.

It is a further object to provide a method of manufacture of cell populations and cell therapy which reduces the risk of teratomas and neoplasms in cell therapy patients.

### SUMMARY OF THE INVENTION

In accordance with a first aspect of the invention, there is provided a method for determining the presence and/or level of contamination by PSC contaminants in a PSC-derived cell population for further use, the method comprising assaying a sample of the PSC-derived cell population against one or a panel of two or more pre-determined non-coding RNAs known or determined to be differentially expressed in PSC contaminants.

In a second aspect of the invention, there is provided a method of lot release of a PSC-derived cell population comprising carrying out the method for determining contamination by PSC contaminants of PSC-derived cell populations as defined above and in dependence on a determination of no or an acceptable level of contamination, releasing said population of PSC-derived cells for further use.

In a third aspect of the invention, there is provided use of non coding RNA expression data or expression profiles for one or a panel of two or more pre-determined non-coding RNAs known or determined to be differentially expressed in PSC contaminants to determine the presence and/or level of contamination by PSC contaminants in a PSC-derived cell population for further use.

In a fourth aspect of the invention, there is provided a kit comprising one or two or more PCR primers each comprising a nucleotide sequence characteristic of a pre-determined non-coding RNA known or determined to be differentially expressed in PSC contaminants, for use in quantitatively determining the amount or expression level of the corresponding one or two or more non-coding RNAs in a sample of PSC-derived cells.

In a fifth aspect of the invention, there is provided a method for detection of contamination in a PSC-derived cell population, the method comprising amplifying and measuring the levels of cDNA molecules comprising nucleotides complementary to a target non-coding RNA known or determined to be differentially expressed in PSC contaminants, which cDNA molecules are derived from non-coding extracted from a sample of the PSC-derived cell population.

In a sixth aspect of the invention, there is provided a method of lot release of a PSC-derived cell population comprising carrying out the method for determining contamination by PSC contaminants of PSC-derived cell populations as defined above and in dependence on a determination of no or an acceptable level of contamination, releasing said population of PSC-derived cells for further use.

In a seventh aspect of the invention, there is provided a PSC-derived cell population for use in cell therapy, the population comprising PSC contaminants of ten cells or fewer per million derived cells as determined by the method above.

In an eighth aspect of the invention, there is provided a method of producing a PSC-derived cell population for use in cell therapy, the method comprising:
inducing differentiation of pluripotent stem cells (PSCs) into derived cells to provide a PSC-derived cell population;
taking a sample of the PSC-derived cell population
subjecting the sample to the method above to determine the presence and level of PSC contaminants in the cell sample
in dependence of determination of presence and level of PSC contaminants being at or less than a pre-determined contamination level, characterizing the PSC-derived cell population as suitable or available for cell therapy; and optionally
administering the PSC-derived cells to a patient in need thereof.

In a ninth aspect of the invention, there is provided a method of treating a patient in need thereof by cell therapy with PSC-derived cells the method comprising producing a PSC-derived cell population for use in cell therapy in accordance with the method above, the method further comprising administering at least a portion of the PSC-derived cell population to the patient.

In a tenth aspect of the invention, there is provided a method for reducing the risk of teratomas arising from PSC-derived cell therapy, the method comprising producing a PSC-derived stem-cell population for use in the cell therapy according to the method above and administering at least portion of the cell population to a patient in need thereof.

In an eleventh aspect of the invention, there is provided use of non coding RNA expression data or expression profiles for one or a panel of two or more pre-determined non-coding RNAs known or determined to be differentially expressed in PSC contaminants to reduce the risk of teratomas in PSC-derived cell therapy.

In a twelfth aspect of the invention, there is provided a system comprising: a set of polynucleotides for detecting at least one or two or more pre determined non-coding RNAs known or determined to be differentially expressed in PSC contaminants and RNAs extracted from a sample from a PSC-derived cell population or cDNAs reverse transcribed from RNAs extracted from a sample from a PSC-derived cell population.

### BRIEF DESCRIPTION OF THE DRAWINGS

FIG. 1 shows, in particular, Microarray data: Expression intensities of miRNAs expressed in iPSCs but not expressed in dpMSCs in the form of a boxplot with whiskers;

FIG. 2 illustrates QRT-PCR data. Confirmation by QRT-PCR of a selected set of miRNAs (from the 39 miRNA panel) that are only expressed in iPSCs;

FIG. 3 shows normalised expression intensities of a selected subset of miRNAs (confirmed by QRT-PCR) expressed in iPSCs, ESCs and non-pluripotent cells;

FIG. 4 shows relative expression of the selected subset 10 miRNA panel in the three independent cell-spike samples'

FIG. 5 shows ddPCR data using the optimised assay with the four selected and prioritised miRNAs, with 100 iPSCs, 10 iPSCs and no iPSCs cell-spiked samples, showing detection of residual contaminating iPSCs in dp MSCs;

FIG. 6 shows ddPCR data using the optimised assay with the four selected and prioritised miRNAs, with 10 iPSCs and no iPSCs cell-spiked samples cell-spiked samples, showing detection of residual contaminating iPSCs in dp MSCs; and

FIG. 7 shows ddPCR assay detection sensitivity in samples containing 10, 5 or 1 iPSC per million MSCs.

### ADVANTAGES OF THE INVENTION

The methods, use and systems of the present invention enable the safe use of PSC-derived cell populations in cell therapy by providing appropriate lot release and in particular an assay or assessment of the presence and level of PSC contaminants in the PSC-derived cell population, upon which a decision to use in cell therapy can be made. In particular, the method enables detection of residual PSC cell contamination of a derived cell population at a level of 10 or fewer residual contaminating PSC cells in a background of one million cells and even 5 or fewer residual contaminating PSC cells in a background of one million.

### DETAILED DESCRIPTION OF THE INVENTION

The invention concerns the use of non-coding RNA expression data, expression levels or expression profiles to determine the presence and/or level of contamination by pluripotent stem cell (PSC) contaminants in a PSC-derived cell population for further use, such as, typically cell therapy. The non coding RNA expression data, expression levels or expression profiles (hereinafter used interchangeably) are for one or a panel of two or more pre-determined non-coding RNAs known or determined to be differentially expressed in PSC contaminants (differentially expressed relative to PSC-derived cells).

Thus, the non-coding expression data can be used in a method for determining the presence and/or level of contamination by PSC contaminants in a PSC-derived cell population for further use, such as cell therapy. A sample of the PSC-derived cell population may be assayed against one or a panel of two or more of the pre-determined non-coding RNAs known or determined to be differentially expressed in PSC contaminants.

The one or a panel of two or more pre-determined non-coding RNAs may be considered biomarkers for PSC contaminants in a PSC-derived cell sample.

The term ‘non-coding RNA’ may include miRNA (microRNA) or other non-coding RNA. The term ‘non-coding RNA’ typically refers to RNAs that do not encode a protein and generally encompass classes of small regulatory RNAs. Other non-coding RNAs referred to above may be, for example, small interfering RNA (siRNA), piwi-interacting RNA (pi RNA), small nuclear RNA (snRNA), small nucleolar RNA (snoRNA), extracellular RNA (exRNA), Small Cajal body RNA (scaRNA) and short hairpin RNA (shRNA). Other non-coding RNAs may further comprise transgenic non-coding RNAs which may function as reporters of non-coding RNA expression. Other non-coding RNAs may be episomal and the methods and/or uses described may require initial steps in which episomal DNA is introduced into the cells described herein whereupon the episomal DNA can be transcribed to produce non-coding RNA which constitutes all or part of the profiled non-coding RNA. In one embodiment, the term non coding RNA does not include non-coding RNAs known as teloRNA.

The term miRNA (microRNA) may include miRNA molecules and either or both miRNA precursors and mature miRNAs as is apparent from the context, but are preferably mature miRNAs.

Hereinafter, embodiments of the invention (and further aspects) will be described by reference to miRNAs. Optionally as an alternative to any or all of the embodiments of the invention described hereinafter, the references to miRNA may instead be to non-coding RNA or other non-coding RNA (such as those defied above) where the context allows (e.g. other than when referring to specific miRNAs).

Preferably, the invention is directed determining the presence and/or level of contamination by PSC contaminants in a PSC-derived cell population by way of the level of expression of pre-defined (and preferably characterizing) miRNA relative to a pre-determined contamination level. The predetermined contamination level may be selected according to the further use, such as therapy and more preferably according to the particular cell therapy and doses required.

Pluripotent stem cells (PSCs) may be embryonic stem cells (ESCs) or induced pluripotent stem cells (iPSCs).

PSC contaminants as used herein are considered to be residual PSC cells, undifferentiated and incompletely differentiated PSCs. By incompletely differentiated PSCs, it is meant PSCs that have not completely differentiated to the cell of the derived cell population, but may be at some intermediate stage, such as a multi-potent stage of differentiation. Such PSC contaminants may comprise undifferentiated PSCs or PSC-derived progenitor cells. Optionally, PSC contaminants may be defined as consisting of undifferentiated PSC cells.

The method of the invention preferably comprises assaying or testing a sample of the PSC-derived cell population against the one or panel of two or more miRNAs known or determined to be differentially expressed in PSC contaminants, such as PSCs or pluripotent cells of intermediate stage of differentiation to the PSC-derived cells. It is meant by this that a procedure is carried out to determine (or measure), in the sample of the PSC-derived cell population, the expression level (or pattern) of one or panel of two or more miRNAs known or determined to be differentially expressed in PSC contaminants.

The one or panel of two or more miRNA against which the assays are carried out may be referred to herein, interchangeably, as a ‘panel’ or ‘target’ miRNAs.

Preferably, in order to determine presence or level of contamination of PSC-contaminants in the PSC-derived cell population, the miRNA expression of the target miRNAs of the sample of the PSC-derived cell population is determined relative to a pre-determined contamination level and/or relative to a background and/or control sample.

A pre-determined contamination level may be selected according to the needs of the further use of the cells, such as in cell therapy. For example, for cell therapy applications requiring a relatively low dose of cells to be administered, an acceptable pre-determined contamination level may be relatively high. On the other hand, where relatively high doses of cells are required for a cell therapy, an acceptable pre-determined contamination level may be relatively low, especially for example if it is to be administered to tissue having greater susceptibility to formation teratomas or neoplasms. Optionally, a pre-determined contamination level may be determined according to an infusion dose of cells having no more than 150 PSC contaminant cells, optionally no more than 100.

Preferably, the pre-determined contamination level is less than or equal to 1000 cells per million of the PSC-derived cell population, more preferably less than or equal to 500 cells per million, still more preferably less than or equal to 100 cells per million, still more preferably less than or equal to 50 cells per million, more preferably 20 cells per million and most preferably less than or equal to 10 cells per million of the PSC-derived cell population. The pre-determined contamination level may, optionally, be 9 cells per million of the PSC-derived cell population, or 8 cells per million, or 7 cells per million or 6 cells per million or 5 cells per million or 4 cells per million. Preferably, the pre-determined contamination level is selected to be 5 cells per million. According to this preferred embodiment, a sample would be assayed against this pre-determined contamination level and would be thus considered unsuitable if it had a PSC contaminant cell level of the pre-determined contamination level or higher, such as in this case preferably 5 cells or more.

Preferably the method comprises measuring the target miRNA expression level in a sample of the PSC-derived cell population and comparing with the measurement made for a positive control, which positive control may be a measurement of target miRNA expression in a control sample comprising cells seeded or spiked with a known or pre-determined level of PSC contaminants. Such pre-determined level of PSC contaminants in the control sample preferably corresponds to a pre-determined level (deemed or considered as safe) of PSC contaminants in a PSC-derived cell, such as the pre-defined contamination levels mentioned above. In a preferred embodiment, the level of PSC contaminants in the control sample is 10 cells per million. In a more preferred embodiment, the level of PSC contaminants is 5 cells per million. Preferably, the measurement of target miRNA expression in the control sample is carried out concurrently with the measurement for the sample of PSC-derived cells.

The control sample, which represents a positive control may be defined as a threshold for the deemed safety of a cell population for a further use such as cell therapy and so, for example, for lot release (i.e. of cell population batches or lots to be released for a next phase of processing for use, e.g. in cell therapy). The threshold may be deemed to represent a desired maximum level or may be represent a value that any acceptable measurement most be less than. The control sample preferably has a level of PSC contaminants corresponding with a pre-determined threshold. In one embodiment, a sample of PSC-derived cells will be considered to have a level of contamination less than a pre-determined level or pre-determined threshold when it has a mean measured target miRNA level of less than the mean of measured target miRNAs of the control sample (assuming that the measurements are made more than once, e.g. at least three times), or respectively less than or equal to (if the threshold is an acceptable level). In another embodiment, in order to accommodate statistical error, the sample of PSC-derived cells may be considered to have a level of contamination less than a threshold level if the mean of the target miRNA expression measurements for the PSC-derived cells (or, alternatively the majority of such measurements or all of the measurements) are outside the range of the threshold, which may be for example the mean and the error about the mean, determined by any suitable method such as confidence intervals or standard deviation. Preferably, the threshold level is such that a measurable miRNA expression level for a sample having a non-detectable level of contaminants and/or a level of contaminants less than the threshold level is distinguishable from the threshold level. Preferably, the method provides a determination of the level of contamination by PSC contaminants in a PSC-derived cell population that is a non-detectable level of contaminants or a detectable level of contaminants that is below the threshold level (which may be a range as mentioned above) or a detectable level of contaminants at the threshold (or within a margin of error of the threshold) or a detectable level of contamination above the threshold level, or the determination may provide a discrete cell contamination value that may optionally have a range corresponding to a statistical error about a mean measured value.

Preferably, the method comprises assaying the sample against the target miRNAs with a positive control using a control sample as mentioned above.

The control sample for the positive control may comprise PSC-derived cells that have been further cultured under conditions unfavourable to PSC contaminants (preferably to a point where residual PSC presence is negligible) and then spiked or seeded with a pre-determined level of PSC contaminants. Alternatively, the positive control may comprise an equivalent cell type to the PSC-derived cells but derived from an alternative source such as a somatic cells (e.g. bone marrow cells), optionally grown out or passaged under conditions unfavourable to the source which cells are spiked or seeded with the pre-determined level of PSC contaminants.

Preferably, the method comprises assaying the sample against the target miRNAs with a background control using a background control sample.

The background control sample may comprise a sample of a cell population which are considered to have negligible (e.g. relative to the pre-determined threshold level, e.g. less than 10% of the threshold level or less than 5% or less than 1%) level of contamination and preferably may be considered an uncontaminated sample. Preferably, the background control sample comprises cells that are the similar to or equivalent to the PSC-derived cells, such as cells having a similar differentiation state and/or phenotype. Such a background control sample may comprise a population of PSC-derived cells that have been grown out or passaged under conditions unfavourable to the PSCs from which they are derived.

Alternatively, the background control sample may comprise cells of equivalent cell type to the PSC-derived cells, but from an alternative source of cells, such as somatic cells (e.g. bone marrow cells) which are optionally grown out in conditions unfavourable to the alternative source of cells.

The background control may then be measured for target miRNA expression, preferably concurrently with the sample of PSC-derived cells and preferably also with a positive control sample. Preferably the background signal may be considered as a value (or range determined iteratively or by statistical analysis) corresponding to a non-detectable level of contamination (or negligible level).

Preferably, the method comprises assaying a sample of the PSC-derived cell population, a background control sample and a positive control sample.

Preferably, the method further comprises assaying the sample of the PSC-derived cell population and any background and/or positive control against one or more endogenous non-coding RNA, preferably miRNA, that is non-specific and preferably is not differentially expressed between the PSC-derived cell population and the PSC contaminants. This provides a normalizer or control feature to the assay, which can be used to ensure, for example, that a result of no detected contamination is valid.

The method therefore preferably comprises assaying the sample of the PSC-derived cell population with a positive control and/or a background control against one or a panel of two or more pre-determined miRNAs (target miRNAs) and optionally an endogenous non-coding RNA (preferably endogenous miRNA) as an endogenous control, wherein the assay step comprises treating and analysing the sample and optional positive control to measure a level of the target miRNAs and optionally the endogenous miRNAs, comparing the expression level of target and endogenous miRNAs in the sample and positive and/or background control and determining therefrom the presence and/or level of contamination by PSC contaminants in the sample.

The present invention may preferably be carried out using any suitable assay technique. For example, the method may comprises at least one technique selected from reverse transcription, microarray, PCR, qPCR, next generation sequencing, nuclease protection, a probe hybridization method, pyrosequencing, and primer extension.

Preferably, the method comprises a step of treating an analysing the samples which comprises detecting or measuring the target miRNAs with a primer and/or probe that has a nucleotide sequence substantially complementary to at least part of a sequence of a target miRNA.

Optionally, the miRNA expression measurement uses any one or combination of quantitative RT-PCR, digital PCR, droplet digital PCR, sequencing, Luminex™ nucleic acid assays, or other hybridization-based technique.

As discussed above, it is preferred that a quantitative measure of expression level of target miRNAs is carried out. It is preferred that the assay comprises quantitative RT-PCR, digital PCR or droplet digital PCR.

Preferably, the method utilises droplet digital PCR (ddPCR).

The method may use a single target miRNA or a panel. The panel may have any number of miRNAs, but is preferably up to 20, more preferably up to 10, still more preferably 2 to 6, e.g. 3 or 4 or 5. Optionally, a single miRNA may be used as the marker of PSC contaminants.

Preferably, the target miRNAs have been identified and validated as PSC contaminant-specific miRNAs.

Preferably, the target miRNAs are selected according to a method described herein.

Preferably, the candidate target miRNAs for use in the method of the invention may be identified by use of a global miRNA assay with a set of miRNA which may comprise 100 or more, preferably at least 200 miRNAs. Preferably, the assay is a microarray with a differential expression analysis of global miRNA expression in samples of a PSC-derived cell population and a PSC population. The PSC-derived cell population should preferably be a population that does not have any PSC contaminants or a negligible level thereof and may, optionally, comprise of a cell population such as the background cell population defined above, e.g. comprising a PSC-derived cells that have been grown out in conditions unsuitable for PSCs. Preferably, the microarray is carried out on a commercial miRNA platform such as that available from Agilent Technologies, Inc (e.g. SurePrint Human miRNA microarrays). Those miRNAs that are specifically expressed in the PSC-derived cell sample and not in the PSCs may form a candidate cohort of target miRNAs from which one or two or more miRNAs may be selected for the contamination determination method of the invention.

There is preferably, therefore, provided a method of selecting a target miRNA for use in a method of determining PSC contamination, the method comprising carrying out an assay of a plurality of miRNAs (e.g. at least 100), preferably a microarray such as defined above. Preferably, a candidate cohort of target miRNAs may be validated in terms of differential expression by a suitable quantitative assay method, such as quantitative reverse transcriptase PCR (qRT-PCR). Preferably the candidate cohort of target miRNA comprises up to 100 miRNAs, preferably up to about 50 miRNA.

Preferably, the target miRNA comprising one or a panel of two or more miRNA selected from the following miRNAs: hsa-miR-367-3p, hsa-miR-302a-3p, hsa-miR-302c-3p, hsa-miR-302b-3p, hsa-miR-302a-5p, hsa-miR-302d-3p, hsa-miR-663a, hsa-miR-1323, hsa-miR-373-3p, hsa-miR-363-3p, hsa-miR-205-5p, hsa-miR-96-5p, hsa-miR-512-3p, hsa-miR-372-3p, hsa-miR-302c-5p, hsa-miR-124-3p, hsa-miR-517a-3p, hsa-miR-517b-3p, hsa-miR-150-3p, hsa-miR-520c-3p, hsa-miR-205-3p, hsa-miR-498, hsa-miR-371a-5p, hsa-miR-3149, hsa-miR-630, hsa-miR-371a-3p, hsa-miR-183-5p, hsa-miR-3692-5p, hsa-miR-32-3p, hsa-miR-34b-3p, hsa-miR-4327, hsa-miR-525-5p, hsa-miR-519d-3p, hsa-miR-629-3p, hsa-miR-3141, hsa-miR-518b, hsa-miR-515-3p, hsa-miR-516b-5p and hsa-miR-519b-3p. This is a preferred candidate cohort of target miRNAs.

Optionally, one or more target miRNA for use in the methods of the invention may be selected from the candidate cohort defined above.

Optionally, the candidate cohort may be refined to provide a refined candidate cohort of up to 20, preferably up to 10 miRNAs, based upon one or more further factors, such as by expression level. Optionally, the refined candidate cohort comprises those miRNAs that are highest expressed in the candidate cohort, e.g. the highest 20 expressed or highest 10 expressed or highest 6 expressed. In one preferred embodiment, the target miRNAs, which may have been selected as the highest expression levels from the candidate cohort, preferably comprise one or more miRNAs selected from the following miRNAs: hsa-miR-302a-3p, hsa-miR-302b-3p, hsa-miR-302c-3p, hsa-miR-302d-3p, hsa-miR-367-3p, hsa-miR-371a-3p, hsa-miR-372-3p, hsa-miR-373-3p, hsa-miR-373-3p, hsa-miR-512-3p and hsa-miR-520c-3p. This represents an example of a refined candidate cohort of target miRNAs.

Preferably, the candidate cohort or refined candidate cohort may be validated in terms of its sensitivity at the desired threshold expression level, and preferably this is validated for the assay method to be carried out. For example, where a desired threshold level of say 10 cells in a million PSC-derived cells is sought, the candidate cohort or refined candidate cohort is preferably subject to a sensitivity analysis at that threshold. This can preferably be achieved by subjecting a set or subset of miRNA (e.g. a refined candidate cohort) to a sensitivity analysis. Preferably, for a PCR-based assay, the sensitivity analysis comprises PCR and preferably one of qRT-PCR or ddPCR or other quantitative and preferably digital PCR methodology suitable for assaying miRNAs.

According to this preferred embodiment, the selected assay method is carried out against a spiked cell sample of known contamination level, the spiked cell sample comprising PSCs in cells corresponding to the PSC-derived cells (e.g. background cells as defined above). Optionally, this quantitative assay is carried out with multiple different concentrations of PSC contaminant-spiked cell samples whereby the sensitivity of the assay using each miRNA in the set (e.g. refined candidate cohort) can be assessed. Those miRNA that are shown to be capable of distinguishing (that is where the differential expression is detectable) at the desired threshold expression level may be selected as a sensitivity-validated panel of target miRNAs to be used in an assay or from which one re more target miRNAs may be selected for use.

In one preferred embodiment, the one or a panel of two or more pre-determined non-coding RNAs comprise one or more miRNAs selected from the following miRNAs: hsa-miR-302a-3p, hsa-miR-302b-3p, hsa-miR-302c-3p, hsa-miR-302d-3p and hsa-miR-367-3p. This is an example of a sensitivity-validated panel of target miRNA which may be used or from which one or more miRNAs may be selected for use in a method of determining contamination levels according to the present invention.

The method of selecting a miRNA or panel thereof may further comprise undertaking an optimization step on the assay method of choice, preferably ddPCR, and carrying out a sensitivity analysis of the sensitivity validated (or other set) candidate cohort of target miRNA. Examples of optimizations that can be carried out are discussed below.

In one preferred embodiment, the one or a panel of two or more pre-determined non-coding RNAs comprise one or more miRNAs selected from the following miRNAs: hsa-miR-302a-3p, hsa-miR-302b-3p, hsa-miR-302c-3p and, hsa-miR-302d-3p, which may preferably be considered a dd-PCR optimized panel of miRNA. Preferably, a miRNA for use alone or in a panel of target miRNAs for use in a method for detecting contamination of PSC contaminants in a PSC-derived cell population using ddPCR in pursuit of a sensitivity of 10 cells per million or less is hsa-miR-302b-3p.

In one embodiment, an endogenous miRNA for use as an endogenous control is selected from one or both of hsa-miR-107 and hsa-miR-130a-3p.

In one preferred embodiment of the invention, the method and system comprises an assay that utilises a quantitative PCR, such as qRT-PCR and most preferably ddPCR. Thus, use of ddPCR in detecting contamination of a PSC-derived cell population by PSC contaminants by assaying against one or more differentially expressed target miRNAs (and optionally an endogenous control), optionally with a background control and a positive control as defined above, is provided in a further aspect and preferred embodiment. Preferably the assay is capable of detecting PSC contaminants to 10 or fewer cells per million and more preferably to 5 cells per million. Preferably the target miRNA has been selected as sensitive at a desired threshold level. Preferably the ddPCR has been optimised for sensitive detection and discrimination at the desired threshold level of the selected one or more target miRNAs. Preferably, the PCR system and method (preferably the ddPCR system and method) is optimized according to one or more of the following:

The reverse transcriptase complementary DNA (RT cDNA) synthesis step is optimized for RNA input and/or RT cDNA primer concentrations The PCR step is optimized for one or more of cDNA concentration, PCR primer concentrations and annealing temperature.

More preferably, the method is droplet ddPCR and the method is optimised for RNA input and RT cDNA primer concentrations. Preferably, the PCR step is also optimised and is preferably optimised for cDNA concentration and PCR primer concentrations and preferably also annealing temperature.

In a particularly preferred embodiment, the method comprises carrying out ddPCR on a sample of PSC-derived cells (and optionally a background control sample and a positive control sample), which preferably comprises extracting total RNA, carrying out a reverse transcription step on target miRNA (e.g. using probes or primers for target miRNA) to generate cDNA corresponding to the target miRNAs, amplifying the cDNA via ddPCR and determining from the amplified signals the expression level of target miRNAs in the respective samples.

Preferably, the method comprises a ddPCR assay having a miRNA reverse transcriptase component in a miRNA RT assay (preferably a TaqMan miRNA assay or similar and following the general protocol provided) and a ddPCR component (preferably using a commercially available droplet digital PCR system and following the general protocol provided).

In a particularly preferred embodiment, the method and system of the invention comprises the use of the aforementioned preferred target miRNAs (e.g. a miRNA selected from the preferred candidate cohort, more preferably an expression validated candidate cohort, still more preferably a sensitivity validated target miRNAs and more preferably the referred to preferred 5, or 4 or 1 target miRNA) in a PSC contaminant contamination assay of PSC-derived cells, preferably by ddPCR which is more preferably optimised as discussed above.

Still more preferably the threshold level of contamination is selected to be up to 20 cells per million, still more preferably up to 15 cells per million and most preferably is 10 cells per million, and more preferably from 5 cells per million to 10 cells per million, preferably 5 cells per million.

The method of the present invention preferably has a low false positive rate and low false negative rate at a threshold of 10 in a million cells, more preferably at a threshold of 5 in a million cells, and more preferably as is the case in preferred embodiments of the method, no false positive results or false negative results. Thus, the method is a highly sensitive and highly specific method.

Another aspect of the invention is directed toward a kit, which preferably comprises one or two or more PCR primers each comprising a nucleotide sequence characteristic of a pre-determined non-coding RNA known or determined to be differentially expressed in PSC contaminants, for use in quantitatively determining the amount or expression level of the corresponding one or two or more miRNAs in a sample of PSC-derived cells. The kit is preferably for use in ddPCR. Preferably the miRNAs are as those indicated as preferred above.

In another aspect of the invention, there is a system comprising: a set of polynucleotides for detecting at least one or two or more pre-determined target miRNAs known or determined to be differentially expressed in PSC contaminants and RNAs extracted from a sample from a PSC-derived cell population or cDNAs reverse transcribed from RNAs extracted from a sample from a PSC-derived cell population. The system preferably comprises an apparatus or system for ddPCR.

The PSC-derived cell population is typically a cell population that it is intended to use for cell therapy (a derived cell product) and can be any cell type derivable from a PSC. Such PSC-derived cells may be, for example, mesenchymal stem cells (MSCs). Preferably, the PSC-derived cells are absent PSC key characteristics. Preferably, the PSC-derived cells are not pluripotent. Optionally, the PSC-derived cells may be multi-potent and of limited lineage. Optionally, the PSC-derived cells may be PSC-derived cardiac cells, retinal cells, neural cells, hepatic cells, etc. Optionally, the PSC-derived cells may be neural stem cells, cardiopoietic cells, etc. Optionally, the PSC-derived cells may be T-cells or CD4+ cells. Preferably, the PSC-derived cells (other than MSCs) are terminally differentiated.

In one embodiment, the PSC-derived cells are MSCs. In one embodiment, the PSC-derived cells are cardiomyocytes. In one embodiment the PSC-derived cells are dopaminergic neurone cells.

In one embodiment, the PSC cells are iPSCs.

Preferably, the PSC-derived cell population is for use in cell therapy.

In one embodiment, the PSC-derived cell population comprises MSCs for use in cell therapy, such as Graft Versus Host Disease, cardiac therapy, tissue revascularization, stroke recovery, Type I and Type II diabetes, kidney disease and kidney transplant recovery, and Alzheimer's Disease.

In another embodiment, the PSC-derived cells are cardiomyocytes for cardiovascular indications.

In another embodiment, the PSC-derived cells are dopaminergic neurone cells for treatment of Parkinson's Disease.

Optionally the PSC-derived cells are CD34+ cells or T-cells for use in cell therapy.

There is provided in a further aspect of the invention, a PSC-derived cell population for use in cell therapy, the population comprising PSC contaminants of at or below a pre-defined threshold, such as ten cells or fewer per million, more preferably at or below a pre-defined threshold in the range from 5 cells to 10 cells million, such as 5 cells per million, as determined by the above methods.

Further, there is provided, a method of producing a PSC-derived cell population for use in cell therapy, the method comprising: inducing differentiation of pluripotent stem cells (PSCs) into derived cells to provide a PSC-derived cell population; taking a sample of the PSC-derived cell population; subjecting the sample to the method as defined above to determine the presence and level of PSC contaminants in the cell sample and in dependence of determination of presence and level of PSC contaminants being at or less than a pre-determined contamination level, characterizing the PSC-derived cell population as suitable or available for cell therapy; and optionally administering the PSC-derived cells to a patient in need thereof. Preferably, the pre-determined contamination level is ten PSC contaminant cells per million of the sample, more preferably five PSC contaminant cells per million of the sample.

In another aspect, there is provided a method of treating a patient in need thereof by cell therapy with PSC-derived cells the method comprising producing a PSC-derived cell population for use in cell therapy in accordance with the above method, the method further comprising administering at least a portion of the PSC-derived cell population to the patient.

In another aspect, there is provided a method for reducing the risk of teratomas arising from PSC-derived cell therapy, the method comprising producing a PSC-derived stem-cell population for use in the cell therapy according to the above method and administering at least portion of the cell population to a patient in need thereof.

In a further aspect of this invention, there is provided a method of treatment of a human or animal patient in need thereof, the method comprising administering one or a plurality of cell therapy doses to said patient, said cell therapy dose effective in treating said patient with a reduced risk of formation of teratomas and/or neoplasms by the cell therapy doses comprising a population of PSC-derived cells characterized as having a PSC contaminant level of below a pre-defined threshold, such as ten cells in a million or five cells per million.

There is thus further provided a use of miR A expression data or expression profiles for one or a panel of two or more pre-determined miR As known or determined to be differentially expressed in PSC contaminants to reduce the risk of teratomas in PSC-derived cell therapy.

The following aspects concern the determination of contamination by source cell contaminants in a source cell-derived cell population for further use.

In one further aspect, there is provided a method for determining the presence and/or level of contamination by source cell contaminants in a source cell-derived cell population for further use, the method comprising assaying a sample of the source cell-derived cell population against one or a panel of two or more pre-determined non-coding RNAs known or determined to be differentially expressed in source cell contaminants.

In another further aspect, there is provided a method of lot release of a source cell-derived cell population comprising carrying out the method for determining contamination by source cell contaminants of source cell-derived cell populations as defined above and in dependence on a determination of no or an acceptable level of contamination, releasing said population of source cell-derived cells for further use.

In a further aspect of the invention, there is provided use of non coding RNA expression data or expression profiles for one or a panel of two or more pre-determined non-coding RNAs known or determined to be differentially expressed in source cell contaminants to determine the presence and/or level of contamination by source cell contaminants in a source cell-derived cell population for further use.

In a further aspect of the invention, there is provided a kit comprising one or two or more PCR primers each comprising a nucleotide sequence characteristic of a pre-determined non-coding RNA known or determined to be differentially expressed in source cell contaminants, for use in quantitatively determining the amount or expression level of the corresponding one or two or more non-coding RNAs in a sample of source cell-derived cells.

In a further aspect of the invention, there is provided a method for detection of contamination in a source cell-derived cell population, the method comprising amplifying and measuring the levels of cDNA molecules comprising nucleotides complementary to a target non-coding RNA known or determined to be differentially expressed in source cell contaminants, which cDNA molecules are derived from non-coding extracted from a sample of the source cell-derived cell population, wherein the target miRNA.

In a further aspect of the invention, there is provided a method of lot release of a source cell-derived cell population comprising carrying out the method for determining contamination by source cell contaminants of source cell-derived cell populations as defined above and in dependence on a determination of no or an acceptable level of contamination, releasing said population of source cell-derived cells for further use.

In a further aspect of the invention, there is provided a source cell-derived cell population for use in cell therapy, the population comprising source cell contaminants of ten cells or fewer per million derived cells, more preferably five cells or few per million, as determined by the method above.

In further aspect of the invention, there is provided a method of producing a source cell-derived cell population for use in cell therapy, the method comprising:
inducing differentiation of pluripotent or multipotent source cells into derived cells to provide a source cell-derived cell population;
taking a sample of the source cell-derived cell population
subjecting the sample to the method above to determine the presence and level of source cell contaminants in the cell sample
in dependence of determination of presence and level of source cell contaminants being at or less than a pre-determined contamination level, characterizing the source cell-derived cell population as suitable or available for cell therapy; and optionally
administering the source cell-derived cells to a patient in need thereof.

In a further aspect of the invention, there is provided a method of treating a patient in need thereof by cell therapy with source cell-derived cells the method comprising producing a source cell-derived cell population for use in cell therapy in accordance with the method above, the method further comprising administering at least a portion of the source cell-derived cell population to the patient.

In a further aspect of the invention, there is provided a system comprising: a set of polynucleotides for detecting at least one or two or more pre-determined non-coding RNAs known or determined to be differentially expressed in source cell contaminants and RNAs extracted from a sample from a source cell-derived cell population or cDNAs reverse transcribed from RNAs extracted from a sample from a source cell-derived cell population. In these further aspects, source cells are cells which may form the source of a cell population for further use, such as in cell therapy, which cell population for further use is derived from the source cell. The derivation may comprise being differentiated therefrom or otherwise derived such as by transformation through uptake of genetic information, or other change. Preferably, both the source cell and the derived cell may be stable, replicable and have identifiable phenotypes. Preferably, the source cell is a stem cell, such as an adult somatic stem cell (e.g. bone marrow, adipose tissue, peripheral blood, skeletal muscle, endometrium, placenta, Wharton's jelly or dental pulp) or are human embryonic stem cells, such as derive from umbilical cord blood or umbilical cord, or are induced pluripotent stem cells or cells derived from pluripotent cells.

Optionally the cells are tissue-specific progenitor cells.

Preferably the cells are human cells. Preferably the cells are stem cells or T-cells.

The cells, especially stem cells, may be sourced from one or multiple sources, which may be, for example, bone marrow, adipose tissue, peripheral blood, skeletal muscle, endometrium, placenta, umbilical cord blood, umbilical cord, Wharton's jelly, dental pulp and cells derived from pluripotent cells.

Further features of these further aspects, including the method of determining a target miRNA panel or biomarker, described above in relation to the system, use and methods concerning PSC-derived cells apply also to these further aspects where the context allows.

References herein to miRNAs and to particular miRNAs use miRNA ID codes (miRNA identifiers) following the convention on naming described on www.mirbase.org. a registry and database of miRNAs managed by the Griffiths-Jones lab from the University of Manchester with funding from the BBSRC. The miRNA identifiers are those valid at 18 Jan. 2018.

### EXAMPLES

Examples are given that show the process for identifying and characterising miRNA for use as specific and sensitive candidates for PSC contamination detection in derived cell samples and to demonstrate the sensitivity achieved with a preferred assay procedure. All cells used for data generation were human cells.

### Methods and Materials

Generation of iPSC-Derived MSC Samples

To minimise the potential for residual iPSCs in the iPSC-derived MSC samples, all the MSCs derived from the iPSCs used here were generated in a way to minimise any potential iPSC contamination by extending their cultivation time in MSC culture medium in which any residual iPSCs would not propagate. This approach would not be possible during routine manufacture of derived MSCs for therapeutic use, but was used here to minimise any potential iPSC contamination. For the purpose of distinguishing over iPSC-derived MSCs that may be used for therapeutic purposes, these derived MSCs shall be referred to as derived propagated MSCs (or dp MSCs).

### Generation of Cell-Spiked Samples

To carry out analysis to determine miRNAs differentially expressed in iPSC versus MSCs derived therefrom, a population of iPSC-derived dp MSCs were prepared with low iPSC contamination and a selection of iPSC spiked samples of iPSC-derived dp MSCs were prepared.

Three independent sets of cell-spiked samples (A, B and C), comprising dp MSCs containing iPSCs spiked-in at known quantities, were generated by serial dilution. The cell-spiked samples comprised 10000 iPSCs, 1000 iPSCs, 100 iPSCs and 10 iPSCs, each seeded into 1 million dp MSCs, respectively. These samples were used for detection sensitivity assessment in both quantitative reverse transcriptase PCR (QRT-PCR) analyses and the droplet digital PCR (ddPCR) analyses described below.

### Generation of Cell Pellets

All cells analysed were human cells. Cell pellets were generated from cells in the following manner:

After centrifugation of the 1×106 cell aliquots, the supernatant was removed by pipette and the cells resuspended in 5 mL of ice-cold phosphate buffer saline pH 7.0 (PBS) and centrifuged at 300×g for 5 min at 4° C. This step was repeated and the cells resuspended in 1 mL of ice-cold PBS, transferred to a 1.5 mL microfuge tube. This was centrifuged at 300×g for 5 min at 4° C. and the supernatant removed by pipette. The resulting cell pellet was further centrifuged 300×g for Imin at 4° C. to collect any residual PBS. This was removed by pipette and the cell pellet snap-frozen in liquid nitrogen and stored at −80° C. until used for RNA isolation within two weeks of generation.

### Extraction of RNA

Total RNA (containing all RNA species, including small non coding RNAs such as miRNAs) was isolated from the cell pellets from cryopreserved cells using the Exiqon miRCURY™ RNA Isolation Kit-Cell & Plant (cat. no. 300110) according to the manufacturer's instructions v2.2. Total RNA concentrations were measured using a Nanodrop™ 1000 spectrophotometer. RNA purity and quality were assessed as ‘pure’ based on 260/280 nM and 260/230 nM ratios and ‘high’ RNA Integrity Numbers (RINs) generated using an Agilent TapeStation 2200.

### MicroRNA Expression Analysis-Microarray

For miRNA expression analysis using human microarrays, aliquots of each cell sample RNA were diluted to 50 ng per mt using nuclease-free water and stored at −80° C. until analysed. Samples were analysed on the Agilent Technologies, Inc. miRNA microarray platform (SurePrint G3 Human v16 microRNA 8×60K microarray slides; miRBase vl6.0, cat no. G4870A) following the manufacturer's instructions v1.7. Briefly, 100 ng of total RNA, from a working solution at 50 ng/ul in nuclease-free water, were used as input for each microarray experiment. Each slide contains 8 individual arrays, each array represents 1,349 microRNAs; 1205 human (mapped to 1194 miRNAs miRbase v20) and 144 viral.

The four key steps of the microarray process were:
1. Labelling of RNA with single-colour, Cy3-based reagent
2. Hybridisation of the labelled RNA samples to the microarray
3. Wash steps
4. Slide scanning, data capture and feature extraction (matching array spots to miRNA IDs) and quality control checks on the resultant image and data files

### Microarray Data Quality Control, Pre-Processing and Normalisation

All microarray data passed Agilent quality control metrics (‘good’ to ‘excellent’). Microarray data pre-processing and normalisation was then carried out with the AgiMicroRNA package in Bioconductor.

Array quality control was performed using outlier testing based on the following metrics:
average signal per array
average background per array
% present (% of miRNAs where expression is detected on each array)
data distributions per sample and pairwise

### Confirmation of Differential Expression by Quantitative Reverse Transcriptase PCR

To confirm the differential expression pattern of the miRNAs identified in the microarray analysis, QRT-PCR analysis was carried out using the same RNA samples as used for the microarray analysis as described below:

For miRNA expression analysis using QRT-PCR, aliquots of each iPSC sample RNA and dp MSCs sample RNA were diluted to a working concentration of 5 ng/pL using nuclease-free water. Samples were analysed using Exiqon LNA™ primer assays and Roche's Lightcycler® 480 PCR system. The QRT-PCR was performed according to the manufacture's protocol. Briefly, 10 ng RNA was reverse transcribed using Exiqon's LNA primer assay components in a working solution of 1× reaction buffer, 1× RT enzyme and 104 UniSp6 spike-in copies (0.5 μL). The RT cycling conditions were as follows: 60 min at 42° C. and 5 min at 95° C. The cDNA was subsequently diluted 100× and analysed using Exiqon's ExiLENT SYBR® Green mastermix and Roche's Lightcycler® 480 PCR system, according to the manufacturer's protocol. Briefly, 100× diluted cDNA was added to the ExiLENT SYBR® Green mastermix in a 1× working solution and added to Exiqon's Pick&Mix PCR panel arrays.

Expression levels were normalised using small nucleolar RNAs C/D box (SNORDs). Five SNORDs were included as candidate normalisers. SNORD 48 and 38B were selected using the GeNorm algorithm as the least variable. MiRNA data was presented as normalised expression (log 2), calculated as delta Cq by subtracting the geometric mean Cq of the normalisers from the Cq of each miRNA for each sample.

Sensitivity Assessment of Selected Candidate miRNAs Suitable for Assay Development Using Quantitative Reverse Transcriptase PCR

To select candidate miRNAs suitable for assay development for the of detection of residual undifferentiated iPSC contamination, QRT-PCR analysis was carried out using RNA extracted from cell-spiked samples comprising dp MSCs containing iPSCs spiked-in at known quantities generated as described above. The same QRT-PCR procedure described above was used in this analysis.

The ddPCR assay was developed and optimised in a manner that would be understood by and within the capability of a person skilled in the art by assessing the following:
The reverse transcriptase complementary DNA (RT cDNA) synthesis step being optimised for RNA input and RT cDNA primer concentrations
The ddPCR step being optimised for cDNA concentration, ddPCR primer concentrations and annealing temperature
These being confirmed at two independent laboratories with different operators, equipment reagents and samples Sensitivity assessment of selected assay development miRNAs suitable for using the optimised droplet digital PCR assay

Sensitivity assessment of selected assay development miRNAs using the optimised ddPCR assay for the detection of residual contaminating undifferentiated iPSCs was carried out using the same samples used in the QRT-PCR sensitivity assessment analysis.

### Example 1—Microarray Analysis to Identify iPSC-Specific miRNAs

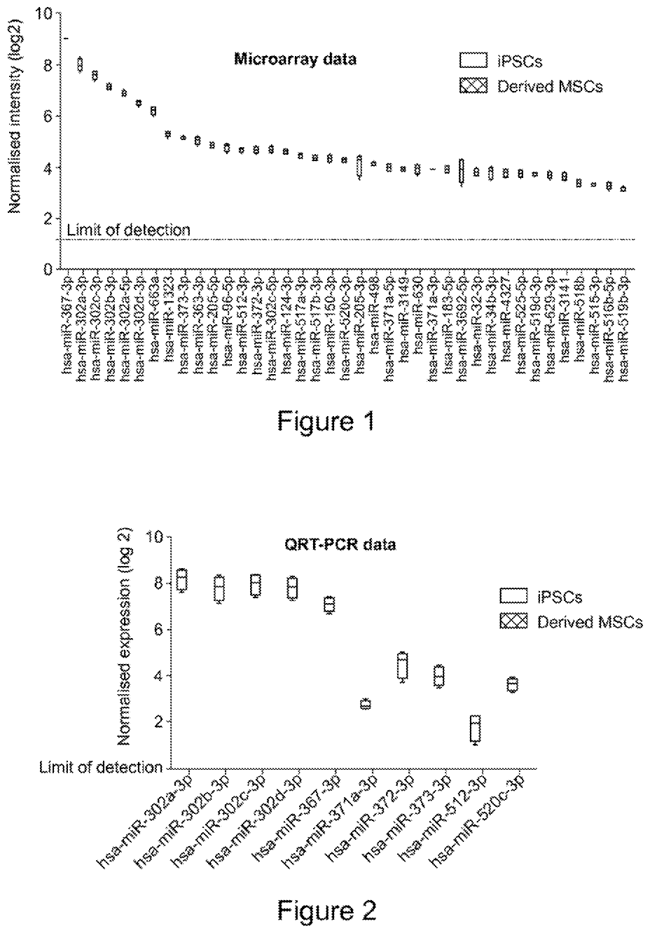

Pure iPSC samples and pure dp MSC samples (generated as described in the Materials and Methods Section above) were profiled for global miRNA expression by microarray in an identification of assay development candidates discovery study. A total of 233 miRNAs were present in the microarray data set. To identify miRNA specifically expressed in iPSCs, but not in the dp MSCs, a differential-expression analysis was carried out to identify those miRNAs that were expressed only in iPSCs. This analysis identified a panel of 39 miRNAs that were expressed only in the iPSCs and not expressed (not detected) in the derived MSCs. The expression intensities and identity of these miRNAs are shown in FIG. 1. FIG. 1 shows, in particular, Microarray data: Expression intensities of miRNAs expressed in iPSCs but not expressed in dpMSCs in the form of a boxplot with whiskers, wherein the black horizontal line within each box represents the median, the boxes range from the 25th percentile to the 75th percentile and the whiskers are min and max of the data [iPSCs, n=4; dp MSCs, n=4].

### Example 2—QRT-PCR Analysis to Confirm Differential Expression of Selected iPSC-Specific miRNAs

To confirm the differential expression pattern of these miRNAs, the same samples that were used for the microarray analysis, were analysed by QRT-PCR using a selected subset (based on expression level) of 10 miRNAs from the 39 miRNA panel. Using this more sensitive and quantitative technology platform, this analysis confirmed the differential expression pattern, i.e. expressed in the iPSCs and not expressed (not detectable) in the dp MSCs, and also rank order of expression of the selected subset miRNA panel. This data is shown in FIG. 2.

In particular, FIG. 2 illustrates. QRT-PCR data. Confirmation by QRT-PCR of a selected set of miRNAs (from the 39 miRNA panel) that are only expressed in iPSCs but not expressed in MSCs. Boxplot with whiskers. The black horizontal line within each box represents the median, the boxes range from the 25th percentile to the 75th percentile and the whiskers are min and max of the data. iPSCs, n=4; DP MSCs, n=4

### Example 3—Microarray Analysis to Confirm Cell-Specificity of Selected iPSC-Specific miRNAs

To examine the utility of the selected subset 10 miRNA panel as assay development candidates for residual contaminating undifferentiated PSCs in derived cell products, their expression in other PSCs and other non-pluripotent potential derived cell types was determined from microarray data. The samples used were: the iPSCs used to identify the iPSC-specific miRNAs (assay development samples; as a comparator), iPSCs from 7 somatic cell types; 5 reprogramming technologies; 3 different culture conditions, ESCs and non-pluripotent cells including (but not exclusive to) MSCs (cord blood-derived, bone marrow-derived, adipose-derived), adipocytes, chondrocytes and osteocytes derived from MSCs, primary adipocytes and chondrocytes, CD34+ cells, primary endothelial cells and Pan and CD4+ T cells. This confirmed that the five of the selected subset miRNA panel i.e. hsa-miR-302a-3p, hsa-miR-302b-3p, hsa-miR-302c-3p, hsa-miR-302d-3p and hsa-miR-367-3p were expressed in all PSC tested and not expressed in any of the non-pluripotent potential derived cell types and were therefore suitable assay development candidates for detection of residual contaminating undifferentiated PSCs in derived cell products. This analysis also showed that the remaining miRNAs in the selected subpanel i.e. hsa-miR-371a-3p, hsa-miR-372-3p, hsa-miR-373-3p, hsa-miR-521-3p and hsa-miR-520c-3p, were not expressed in all the non-pluripotent cells tested, but were not necessarily expressed in all PSCs. This does not necessarily exclude them as assay development candidates for residual contaminating undifferentiated PSCs in derived cell products, but they would have to be further tested for potential as assay development candidates on a case-by-case basis with the particular PSC line being used to generate derived cell product.

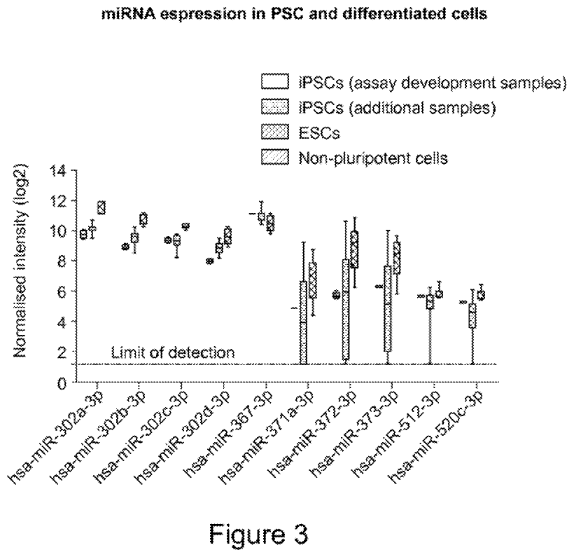

FIG. 3 illustrates the microarray data. In particular, FIG. 3 shows normalised expression intensities of a selected subset of miRNAs (confirmed by QRT-PCR) expressed in iPSCs, ESCs and non-pluripotent cells. Boxplot with whiskers. The black horizontal line within each box represents the median, the boxes range from the 25th percentile to the 75th percentile and the whiskers are min and max of the data. In FIG. 3: iPSCs (assay development samples)=n=4; iPSCs (additional samples) n=40; ESC=n=8; Non-pluripotent cells n=3-8 in each cell type

### Example 4—Sensitivity Analysis by QRT-PCR of a Selected iPSC-Specific miRNAs Using Cell-Spiked Samples

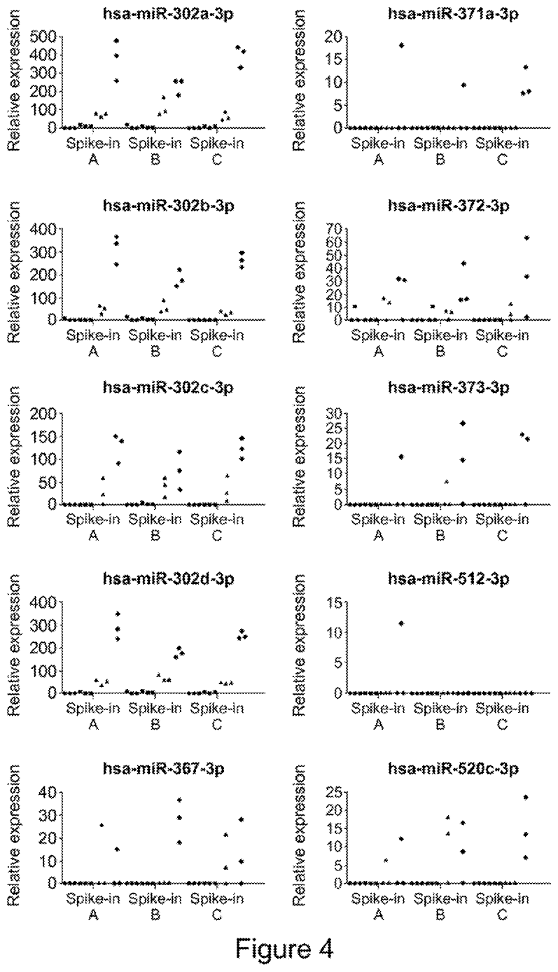

To assess the sensitivity of the selected subset 10 miRNA panel (those confirmed by QRT-PCR) for assay development candidates for residual contaminating undifferentiated PSCs in derived cell products, their detection sensitivity was assessed in three independent cell-spiked samples comprising 10000 iPSC, 1000 iPSC, 100 iPSCs and 10 iPSCs, each seeded into 1 million dp MSCs, respectively. FIG. 4 shows relative expression of the selected subset 10 miRNA panel in the three independent cell-spike samples. This analysis showed that of the selected subset 10 miRNA panel, four miRNAs i.e. hsa-miR-302a-3p, hsa-miR-302b-3p, hsa-miR-302c-3p, hsa-miR-302d-3p, had the highest detection sensitivity potential as assay development candidates for the detection of very low numbers of residual contaminating undifferentiated PSCs in derived cell products, being able to detect between 100 and 10 iPSCs in 1 million derived MSCs.

In FIG. 4: QRT-PCR analysis of the selected subset 10 miRNA panel assay development candidates with the A, B and C cell-spiked samples presented as Log 2 normalised relative expression values

Diamonds=10000 iPSCs seeded into 1 million dp MSCs; Triangles=1000 iPSC seeded into 1 million dp MSCs; Squares=100 iPSCs seeded into 1 million dp MSCs; Circles=10 iPSCs seeded into 1 million dp MSCs

### Example 5—Development of a Highly Sensitive Droplet Digital PCR Assay with a Selected Panel of iPSC-Specific miRNAs

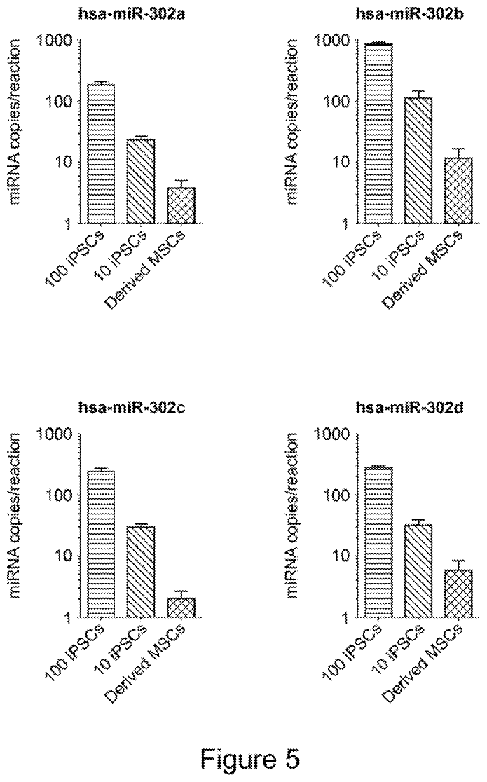

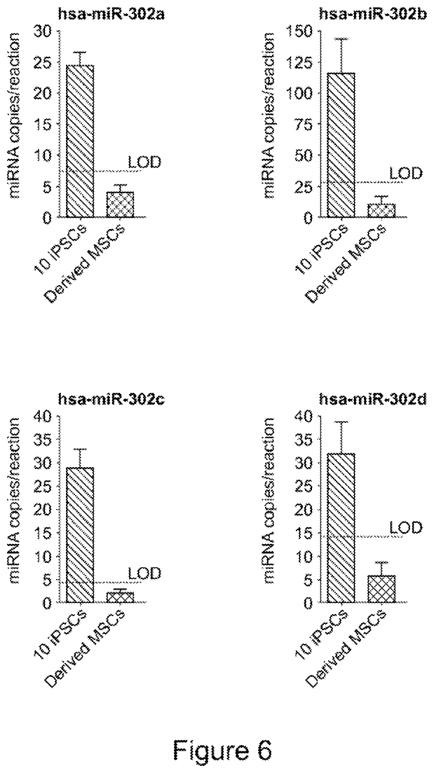

Based on the microarray cell-specificity data (FIG. 3) and the QRT-PCR cell spiked sample detection sensitivity data (FIG. 4), the following miRNAs: hsa-miR-302a-3p, hsa-miR-302b-3p, hsa-miR-302c-3p, hsa-miR-302d-3p, were further selected and prioritised assay development candidates for the detection of very low numbers of residual contaminating undifferentiated PSCs in derived cell products. Based on the limitation of detection sensitivity achieved by QRT-PCR (FIG. 4) for these miRNAs with the cell-spiked samples, it was decided to migrate to a more sensitive technology platform, with absolute quantification capability i.e. droplet digital PCR. In conjunction with using these four iPSC-specific assay development candidates, a ddPCR assay was developed, optimised and validated as detailed in the Materials and Methods. Final detection sensitivity, specificity, precision and reproducibility assessment was carried out using the 100 iPSC, 10 iPSC and no iPSC cell-spiked samples. This analysis showed that the optimised assay was able to consistently detect <10 iPSCs in a background of one million MSCs, as shown in FIGS. 5 and 6. In addition, the absolute copies/ddPCR for hsa-miR-302a-3p, hsa-miR-302b-3p, hsa-miR-302c-3p and hsa-miR-302d-3p for a contamination level of 10 iPSCs were well above the estimated limit of detection (LOD) for as indicated in FIG. 6.

FIG. 5 shows: Example ddPCR data using the optimised assay with the four selected and prioritised miRNAs, with 100 iPSCs, 10 iPSCs and no iPSCs cell-spiked samples, showing detection of residual contaminating iPSCs in dp MSCs. Combined data is shown for three independent cell-spiked samples (A, B and C) and is presented as miRNA copies/ddPCR bar plots as mean±SD on a log 10 scale. 100s iPSC=100 iPSCs seeded into 1 million dp MSCs; 10 iPSCs=10 iPSCs seeded into 1 million dp MSCs; DP MSCs=no iPSCs seeded into 1 million MSCs

FIG. 6 shows: Example ddPCR data using the optimised assay with the four selected and prioritised miRNAs, with 10 iPSCs and no iPSCs cell-spiked samples cell-spiked samples, showing detection of residual contaminating iPSCs in dp MSCs. Combined data is shown for three independent cell-spiked samples (A, B and C) and is presented as miRNA copies/ddPCR bar plots as mean±SD on a linear scale. Estimated LOD at 95% confidence indicated by dotted line and calculated as LOD=mean MSC miRNA copies per ddPCR+(3.3 XSD). 10 IPSCs=10 iPSCs seeded into 1 million dp MSCs; DP MSCs=no iPSCs seeded into 1 million dp MSCs

### Example 6—Sensitivity Analysis by ddPCR of a Selected iPSC-Specific miRNAs Using Cell-Spiked Samples

To further assess the sensitivity of the highest sensitivity potential four miRNAs identified above, i.e. hsa-miR-302a-3p, hsa-miR-302b-3p, hsa-miR-302c-3p, hsa-miR-302d-3p, for residual contaminating undifferentiated PSCs in derived cell products, their detection sensitivity was further assessed in three independent cell-spiked samples comprising 10 iPSCs, 5 iPSCs and 1 iPSC, each seeded into 1 million BM-MSCs (bone marrow MSCs) (cell-spiked samples generated by laser capture), respectively and also with 1 million BM-MSCs.

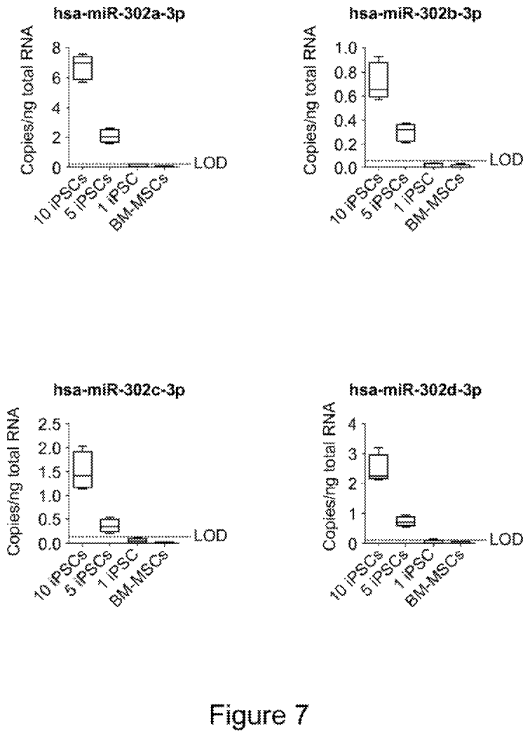

FIG. 7 shows: ddPCR assay detection sensitivity in samples containing 10, 5 or 1 iPSC per million MSCs. Background signal is shown for BM-MSCs. Data is expressed as target miRNA copy numbers/ng RNA. Boxplot with min and max whiskers. Plotted are results two independent cDNA synthesis reactions carried out for each sample, 10 iPSCs (n=2), 5 iPSCs (n=2), 1 iPSC (n=2), BM-MSCs (n=2). Estimated LODs are indicated by a dotted lines and are calculated as follows LOD-LOB+(1.645×Standard deviation of 1 iPSC samples); where LOB=mean of BM-MSC samples+(1.645×Standard deviation of BM-MSC samples).

This analysis shows the ddPCR assay for all four of the targets, hsa-miR-302a-3p, hsa-miR-302b-3p, hsa-miR-302c-3p, hsa-miR-302d-3p, is highly sensitive and can clearly detect 5 iPSCs in 1 million BM-MSCs (above background 1 million BM-MSCs). Calculated LODs were estimated at either equivalent to around 1 iPSC in 1 million BM-MSCs (in hsa-miR-302a-3p, hsa-miR-302c-3p, hsa-miR-302d-3p) or slightly above 1 iPSC in 1 million BM-MSCs (in hsa-miR-302b-3p).

In order to assess the diagnostic accuracy (sensitivity and specificity) of the assay, the total number of tests carried out on samples containing 10 iPSCs and 5 iPSCs in a background of 1 million BM-MSCs and 0 iPSC (1 million BM-MSCs) were compiled and the number of true positives, true negatives, false negatives and false positives determined.

Table-Assessment of Diagnostic ddPCR Assay Sensitivity and Specificity at Different iPSC Inputs

| | | |
|---|---|---|
| Positive | True positive (a) 183 | False positive (b) 0 |
| Negative | False negative (c) | True negative (d) 139 |
| Accuracy | % sensitivity = | % sensitivity = |
| | a/(a + c) × 100 = 100% | d/(b + d) × 100 = 100% |

| | |
|---|---|
| Test | Outcome and Number of Tests |

| Score | 5 PSCs | 0 PSCs |
|---|---|---|
| Positive | True positive (a) 64 | False positive (b) 0 |
| Negative | False negative (c) | True negative (d) 64 |
| Accuracy | % sensitivity = | % sensitivity = |
| | a/(a + c) × 100 = 100% | d/(b + d) × 100 = 100% |

This analysis clearly shows that the ddPCR assay has a 100% sensitivity (no false negatives) and 100% specificity (no false negatives) at both the 10 and 5 iPSC in 1 million BM-MSC detection level.

The invention has been described with reference to a preferred embodiment. However, it will be appreciated that variations and modifications can be effected by a person of ordinary skill in the art without departing from the scope of the invention.

1. A method for determining the presence and/or level of contamination by pluripotent stem cell (PSC) contaminants in a PSC-derived cell population for further use, the method comprising
assaying a sample of the PSC-derived cell population against at least one pre-determined miRNAs known or determined to be differentially expressed in PSC contaminants, where one or a panel of miRNAs one or more miRNAs selected from a group consisting of: hsa-miR-302a-3p, hsa-miR-302b-3p, hsa-miR-302c-3p and, hsa-miR-302d-3p,
wherein the step of assaying the sample of the PSC-derived cell population comprises:
treating the sample to measure a level of the at least one pre-determined miRNAs by quantitative reverse transcriptase polymerase chain reaction (RT-PCR), digital PCR or droplet digital PCR; and
determining therefrom the presence and/or level of contamination by PSC contaminants in the sample;
wherein the PSC contaminants comprise one or more of undifferentiated PSCs and incompletely differentiated PSCs; and
wherein the method is configured to determine whether the level of contamination by PSC contaminants meets the criterion of ten or fewer PSC contaminant cells per one million cells of the sample.

2. The method as claimed in claim 1, wherein the method comprises determining the level of PSC contaminants in a sample of the PSC-derived cell population relative to a positive control of cells seeded or spiked with a known or pre-determined level of PSC contaminants.

3. The method as claimed in claim 2, wherein the positive control comprises a sample of cells seeded or spiked with PSC contaminants in an amount of 10 cells per million.

4. The method as claimed in claim 2, wherein the positive control comprises a sample PSC-derived cells which have been grown out or passaged under conditions where PSC contaminants are negligible and then seeded or spiked with a pre-determined level of PSC contaminants.

5. The method as claimed in claim 1, wherein the one or a panel of two or more pre-determined miRNAs comprise the miRNA hsa-miR-302b-3p.

6. The method as claimed in claim 1, wherein the method further comprises assaying the sample of the PSC-derived cell population against one or more endogenous non-coding RNA that is non-specific or non-differentially expressed between the PSC-derived cell population and the PSC contaminants by way of a normalizer or a control.

7. The method as claimed in claim 1, wherein the endogenous non-coding RNA is a miRNA selected from hsa-miR-107 and hsa-miR-130a-3p.

8. The method as claimed in claim 1, wherein the method comprises assaying a sample of the PSC-derived cell population with a negative control comprising an uncontaminated cell sample.

9. The method as claimed in claim 1, wherein the further use is for cell therapy.

10. The method as claimed in claim 1, wherein the PSC-derived cells are mesenchymal stem cells (MSCs).

11. The method as claimed in claim 1, wherein the PSCs are induced pluripotent stem cells (iPSCs).

12. The method as claimed in claim 2, wherein the positive control comprises a sample of cells seeded or spiked with PSC contaminants in an amount of 1 cell and/or 5 cells and/or 10 cells per million.

13. The method as claimed in claim 1, configured to determine whether the level of contamination by PSC contaminants meets the criterion of five or fewer PSC contaminant cells per one million cells of the sample.

14. The method according to claim 1, wherein the step of treating and analyzing comprises droplet digital PCR (ddPCR).

15. A pluripotent stem cell (PSC)-derived cell population for use in cell therapy, the population comprising PSC contaminants of ten cells or fewer per million as determined by the method of claim 1.

16. The PSC-derived cell population for use in cell therapy, the population comprising PSC contaminants of five cells or fewer per million as determined by the method claim 1.

17. A method of producing a pluripotent stem cell (PSC)-derived cell population for use in cell therapy, the method comprising:
inducing differentiation of PSCs into derived cells to provide a PSC-derived cell population;
taking a sample of the PSC-derived cell population;
determining the presence and level of PSC contaminants in the cell sample;
in dependence of determination of presence and level of PSC contaminants being at or less than a pre-determined contamination level, characterizing the PSC-derived cell population as suitable or available for cell therapy; and optionally
administering the PSC-derived cells to a patient in need thereof.
